(19) United States
(12) Patent Application Publication
(10) Pub. No.: US 2019/0069544 A1
(43) Pub. Date: Mar. 7, 2019

(54) COMPOSITION COMPRISING A MEL, A FATTY ACID METHYL ESTER AND A NON-IONIC SURFACTANT HAVING A HLB VALUE GREATER THAN OR EQUAL TO 12

(71) Applicant: OLEON NV, EVERGEM (Ertvelde) (BE)

(72) Inventors: Marion RANDU, CLERMONT (FR); Sylvie HERY, JAUX (FR); Pierre RAVIER, COMPIEGNE (FR); Sophie DEPREY, MARGNY-LES-COMPIEGNE (FR)

(21) Appl. No.: 16/085,357

(22) PCT Filed: Mar. 17, 2017
(86) PCT No.: PCT/EP2017/056464

§ 371 Date: Sep. 14, 2018

(30) Foreign Application Priority Data
Mar. 17, 2016 (FR) 1652280

(51) Int. Cl. A01N 37/12; A01N 25/30; A01N 25/04; A01N 43/16; A01N 35/02; A01N 31/16; A61K 8/06; A61K 8/37; A61K 8/60; A61K 8/86; A61Q 19/00

(52) U.S. Cl. CPC A01N 37/12; A01N 25/30; A01N 25/04; A01N 43/16; A01N 35/02; A61Q 19/00; A61K 8/062; A61K 8/37; A61K 8/602; A61K 8/86; A01N 31/16

(57) ABSTRACT

The present invention relates to a composition comprising at least one fatty acid methyl or ethyl ester, at least one lipid of mannosylerythritol and at least one non-ionic surfactant having an HLB value greater than or equal to 12, to the method for producing same and to the uses thereof, in particular for preparing an emulsion.

The present invention relates to an emulsifiable composition and to an emulsion comprising it. The present invention also relates to a process for the preparation of the composition and of the emulsion according to the invention, and the use of the composition according to the invention, in particular in the preparation of an emulsion.

In the context of the present application, by “emulsifiable composition”, is meant a composition which can be placed in the state of an emulsion, in particular during contact with water. The emulsifiable compositions find an application in numerous fields. It is known in particular to use emulsifiable compositions in the cosmetics industry and in the agricultural industry, in particular for the protection of plants. In the agricultural industry, the emulsifiable compositions, also called emulsifiable concentrates, are generally used in the preparation of phytosanitary mixtures, such as in the form of phytosanitary emulsions, such as for example herbicide, fungicide, insecticide, algicide emulsions or also emulsions for stimulating the defences of plants.

As well as their “emulsifiability” or ability to be placed in the state of an emulsion, the emulsifiable compositions can have other properties. By way of example, in the context of a phytosanitary use, an emulsifiable composition can also have a penetrating, moistening and/or wetting property. An emulsifiable composition having a wetting property promotes the spreading and the retention of the emulsion comprising it on crops. This results, on the one hand, in a reduction of the quantity of phytosanitary emulsion to be sprayed on the crops, and on the other hand, in an improvement in the effectiveness of the phytosanitary emulsions. The use of emulsifiable compositions having wetting properties thus allows a reduction in the efforts and costs associated with the treatment of crops.

Moreover, whatever the types of industry in which they are used, it is preferable that the emulsifiable compositions are environmentally friendly and are less toxic for the operators using them. This is particularly important when they are used in the preparation of phytosanitary emulsions, as these are generally spread over crops in significant quantities so that it is preferable, or even necessary, that the emulsifiable compositions are ecologically advantageous, and in particular biodegradable.

More particularly, it would be useful to develop emulsifiable compositions:
having an excellent emulsifiability,
making it possible to obtain stable emulsions,
having a good ability to increase the wetting power of the emulsions comprising them, such as phytosanitary emulsions, and
which would moreover be environmentally friendly.

The work of the inventor has made it possible to demonstrate that a specific composition had all of the advantageous properties described above.

The invention therefore relates to a composition comprising:
between 75 and 99% by weight of at least one fatty acid methyl or ethyl ester,
between 0.01 and 20% by weight of at least one mannosylerythritol lipid, and
between 0.01 and 20% by weight of at least one non-ionic surfactant having an HLB value greater than or equal to 12,
   the percentages by weight being indicated with respect to the total weight of the composition.

It will be noted that in the context of the present application, and unless stated otherwise, the value ranges indicated are inclusive.

The composition according to the invention comprises between 75 and 99% by weight of at least one fatty acid methyl or ethyl ester, with respect to the total weight of the composition.

Advantageously, the fatty acid methyl or ethyl ester(s) comprised in the composition according to the invention are selected from methyl oleate, ethyl oleate, the methyl or ethyl esters of rapeseed, soya, olive, sunflower, castor, palm and/or linseed oil fatty acids, or mixtures thereof.

Preferably, the total quantity of fatty acid methyl or ethyl ester(s) in the composition according to the invention is comprised between 80 and 98% by weight, with respect to the total weight of the composition.

By total quantity of fatty acid methyl or ethyl ester(s) present in the composition, is meant the quantity of methyl ester molecule(s) or, respectively, ethyl ester molecule(s) of fatty acid(s) present in said composition.

Preferably, the total quantity of fatty acid methyl or ethyl ester(s) in the composition according to the invention is comprised between 85 and 96% by weight, with respect to the total weight of the composition.

Preferably, the fatty acid methyl or ethyl ester(s) comprised in the composition according to the invention are fatty acid methyl or ethyl ester(s) comprising a carbon-containing chain having between 8 and 24 carbon atoms, more preferentially between 12 and 20 carbon atoms, even more preferentially between 16 and 18 carbon atoms.

Preferably, in the composition according to the invention, the fatty acid methyl or ethyl ester(s) are methyl or ethyl esters of rapeseed, soya and/or olive oil fatty acids, more preferentially of rapeseed and/or olive oil fatty acids.

Preferably, the composition according to the invention comprises at least one fatty acid methyl ester.

The composition according to the invention also comprises between 0.01 and 20% by weight of at least one of mannosylerythritol lipid, with respect to the total weight of the composition.

According to the invention, a “mannosylerythritol lipid” (also called “MEL”) is a surfactant belonging to the class of glycolipids. More particularly, a MEL is an amphiphilic molecule the hydrophilic part of which is formed by a mannosylerythritol residue, and the hydrophobic part of which is formed by at least one fatty acid.

More particularly, by “MEL”, is meant a molecule having the following general formula (I):

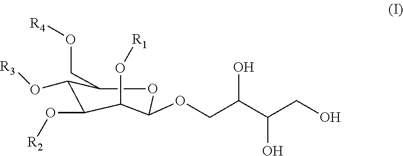

in which:
R₁ and R₂, identical or different, represent an unsaturated or saturated fatty acid, and
R₃ and R₄, identical or different, represent an acetyl group or a hydrogen atom.

Preferably, in the present invention, by “MEL”, is meant a molecule having the following formula (II):

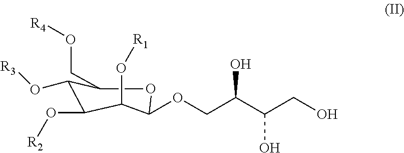

in which:
R₁ and R₂, identical or different, represent an unsaturated or saturated fatty acid, and
R₃ and R₄, identical or different, represent an acetyl group or a hydrogen atom.

The formulae (I) and (II) above can represent several molecules, each molecule therefore being a MEL. By “MELs”, is meant at least two different molecules of formulae (I), and more particularly of formula (II).

The MELs are generally classified in four classes of molecules, denoted A to D, according to their degree of acetylation in positions R₃ and R₄. The class of the MELs-A comprises the molecules of formulae (I) or (II) having two acetyl groups in positions R₃ and R₄. The class of the MELs-B and the class of the MELs-C comprise molecules of formulae (I) or (II) having a single acetyl group in positions R₄ and R₃ respectively. Finally, the class of the MELs-D comprises molecules of formulae (I) or (II) not having an acetyl group (R₃═R₄═H).

As well as by their degree of acetylation, the MELs can vary in their structure, by the nature of the fatty acids which comprise their hydrophobic part. This variation is generally a function of the process implemented for obtaining the MELs.

The MELs are generally obtained by processes implementing the culture of fungi, and more particularly of yeasts.

Advantageously, the MELs to which the present application relates are obtained by a fermentation process, comprising the following steps:

culturing a fungal strain and more particularly a yeast strain in the presence of a carbon source in order to obtain MELs; and

recovering the MELs thus obtained.

The strains from which it is possible to obtain MELs are well known to a person skilled in the art. By way of example, it is known to use fungal strains of the genus Pseudozyma or of the genus Ustilago, in order to obtain MELs.

Advantageously, the strains used in the fermentation process described above, making it possible to obtain MELs, are fungal strains belonging to the genus Pseudozyma. Preferably, the strain is Pzeudozyma antartica or Pzeudozyma aphidis.

Such strains are usually cultured in a reactor in a medium comprising glucose, water and/or salts (such as magnesium sulphate, monopotassium phosphate and/or ammonium nitrate).

Advantageously, the different components of the medium (glucose and strains in particular) are sterilized separately before introduction into the reactor.

The temperature of the medium is preferably comprised between 20 and 35° C., more preferentially between 25 and 30° C.

Advantageously, the carbon source allowing the production of MELs by the strain is an oil, such as a vegetable oil. Preferably, the source of carbon is a soya oil or even more preferentially a rapeseed oil. These oils are particularly rich in fatty acids comprising a carbon-containing chain with 18 carbon atoms, such as oleic, linoleic and/or linolenic acid, as well as, to a lesser degree, in fatty acids comprising a carbon-containing chain with 16 carbon atoms, such as in palmitic acid.

The recovery of the MELs following the culture step can comprise a step of separating the MELs from the other components of the medium. This step can be done by standard separation methods known to a person skilled in the art.

Advantageously, the recovery of the MELs can comprise one or more of the following separation methods:
settling;
evaporation of water or drying;
filtration; and/or
centrifugation.

Advantageously, the total quantity of MEL(s) in the composition according to the invention is comprised between 0.1 and 10% by weight, with respect to the total weight of the composition.

By total quantity of MEL(s) present in the composition, is meant the quantity of molecule(s) of MEL(s) of formulae (I) or (II) present in said composition.

Preferably, the total quantity of MEL(s) is comprised between 0.5 and 6% by weight, more preferentially between 0.8 and 3% by weight, with respect to the total weight of the composition.

Favourably, the composition comprises at least two MELs selected from the group constituted by MEL-A, MEL-B, MEL-C and MEL-D.

Preferably, the composition according to the invention comprises MEL(s)-A, MEL(s)-B, MEL(s)-C and optionally MEL(s)-D, more preferentially MEL(s)-A, MEL(s)-B, MEL(s)-C and MEL(s)-D.

Advantageously, the composition according to the invention comprises MELs-A and MELs-B at a content comprised between 50 and 90% by weight, preferably comprised between 60 and 85% by weight, the percentages by weight being indicated with respect to the weight of the total quantity of MELs.

Advantageously, the composition according to the invention comprises MELs-C at a content greater than or equal to 5% by weight, preferably greater than or equal to 10% by weight, the percentages by weight being indicated with respect to the weight of the total quantity of MELs.

More particularly, the composition according to the invention can comprise MELs-A and MELs-B at a content comprised between 60% and 75% by weight, and MELs-C at a content greater than or equal to 20% by weight, the percentages by weight being indicated with respect to the weight of the total quantity of MELs.

The composition according to the invention also comprises between 0.01 and 20% by weight of at least one non-ionic surfactant having an HLB value greater than or equal to 12.

The HLB value makes it possible to define, and in particular to quantify, the balance between the hydrophilic part and the lipophilic part of a surfactant molecule, this balance being linked to the solubility of the surfactant in water. The HLB value can vary from 0 to 20. The higher the HLB value, the greater the solubility of the surfactant in water.

In the context of the present application, the calculation of the HLB is carried out by Griffin's method:

$$HLB = 20 \times \frac{Molecular mass of the hydrophilic part}{Molecular mass of the molecule}$$

Advantageously, the total quantity of non-ionic surfactant(s) having an HLB value greater than or equal to 12 in the composition according to the invention is comprised between 0.1 and 15% by weight, with respect to the total weight of the composition.

By total quantity of non-ionic surfactant(s) having an HLB value greater than or equal to 12 present in the composition, is meant the quantity of molecule(s) of non-ionic surfactant(s) having an HLB value greater than or equal to 12 present in said composition.

Preferably, the total quantity of non-ionic surfactant(s) having an HLB value greater than or equal to 12 is comprised between 0.5 and 10% by weight, more preferentially between 0.8 and 8% by weight, even more preferentially between 1 and 5% by weight, with respect to the total weight of the composition.

Preferably, the non-ionic surfactant(s) having an HLB value greater than or equal to 12 comprised in the composition according to the invention is/are selected from the group constituted by:
polysorbates (ethoxylated esters of fatty acids and sorbitol), such as polysorbate 20, polysorbate 60 and/or polysorbate 80,
alkyl polyglucosides, such as those having alkyl chains comprising between 4 and 16 carbon atoms,
polyethylene glycol fatty acid esters, and,
polyglycerol fatty acid esters.

Advantageously, the non-ionic surfactant(s) comprised in the composition according to the invention have an HLB (“Hydrophilic-Lipophilic Balance”) value comprised between 12 and 20, preferably between 12 and 18, more preferentially between 12 and 16, even more preferentially between 12 and 14.

Preferably, the non-ionic surfactant(s) comprised in the composition according to the invention have an HLB value greater than or equal to 12.5, more preferentially greater than or equal to 13.

The composition according to the invention is emulsifiable. More particularly, the composition according to the invention has an excellent emulsifiability, i.e. when it is added to water, the composition according to the invention allows the formation of an emulsion:
uniform (complete), and
spontaneously.

By “spontaneously”, is meant that only gentle stirring is required in order to obtain an emulsion. By way of example of gentle stirring, the container into which the composition according to the invention and the water are added can be turned over manually so as to turn through an angle of 180° C., then returned to its initial position, the operation taking approximately two seconds, according to what is indicated in step (i) of the standard CIPAC MT 36.3 (“CIPAC method 2000. Prepared by the German Formulation Panel (DAPF). Chairman: G Menschel.”).

In the context of the present application, any reference made to a standard is a reference to the standard in force at the filing date.

Advantageously, the emulsifiability of the composition according to the invention is characterized according to step i) of the standard CIPAC MT 36.3.

The emulsifiability of the composition according to the invention is more fully described in Example 2.

Moreover, when an emulsion is prepared from the composition according to the invention, it is stable over time.

Moreover, it has been demonstrated by the inventors that, surprisingly, an emulsion comprising the composition according to the invention has a high wetting power, and has a white colour that is particularly favourable to its use in various fields, such as for example in cosmetics. These characteristics are more fully described hereafter.

Advantageously, the composition according to the invention comprises between 0,1 and 20% by weight of at least one alcohol having a number of carbon atoms comprised between 1 and 16, with respect to the total weight of the composition.

By “alcohol”, is meant more particularly a linear or branched alcohol. By “linear or branched”, is meant that the cyclic alcohols are specifically excluded. Even more particularly, the alcohol is an alcohol constituted by a linear or branched hydrocarbon-containing chain, substituted by one or more hydroxyl (OH) group(s). By “hydrocarbon-containing chain”, is meant a chain constituted only by carbon and hydrogen atoms, the hydrocarbon-containing chain then comprising between 1 and 16 carbon atoms. In other words, the alcohol does not comprise a heterosubstituent other than the hydroxyl group(s),

The alcohol(s) comprised in the composition according to the invention also make it possible to improve the stability and the wetting power of the emulsions formed from this composition.

Preferably, the total quantity of alcohol(s) in the composition according to the invention is comprised between 0.5 and 10% by weight, with respect to the total weight of the composition.

By total quantity of alcohol(s) present in the composition, is meant the quantity of molecule(s) of alcohol(s) present in said composition, the molecules of alcohol being as defined above.

Preferably, the total quantity of alcohol(s) is comprised between 0.8 and 8% by weight, more preferentially between 1 and 5% by weight, with respect to the total weight of the composition.

Advantageously, the alcohol is saturated.

The alcohol(s) are therefore linear or branched. As linear alcohols, heptanol (or heptan-1-ol), octanol (also called 15 octan-1-ol or caprylic alcohol), lauric alcohol or nonanol (or nonan-1-ol or pelargonic alcohol) may be mentioned. As branched alcohols, octan-2-ol, 2-ethyl-hexanol, 7-methyl-octan-1-ol or 6-methyl-pentan-1-ol may be mentioned.

Advantageously the alcohol(s) comprised in the composition according to the invention have a hydrocarbon-containing chain comprising a number of carbon atoms comprised between 4 and 14, preferentially between 6 and 12.

Preferably, the alcohol(s) are selected from the alcohols having a hydrocarbon-containing chain comprising 8 to 10 carbon atoms or mixtures thereof. In particular, they are octanol, octan-2-ol, 2-ethyl-hexanol and/or lauric alcohol, preferably octan-2-ol.

Advantageously, the alcohol can be obtained from renewable sources, such as from animal fats or vegetable oils.

In particularly preferred manner, the non-ionic surfactant having an HLB value greater than or equal to 12 present in the composition according to the invention is a polyethylene glycol fatty acid ester.

In the context of the present application, by “polyethylene glycol”, also called “PEG”, is meant a polymer of ethylene oxides having a molar mass of less than 20,000 g·mol⁻¹.

In fact, when the composition according to the invention comprises a polyethylene glycol fatty acid ester, an emulsion prepared from this composition will have a particularly high wetting power. This effect is more fully described in Example 5.

Advantageously, the polyethylene glycol comprised in the polyethylene glycol fatty acid ester has a molar mass comprised between 200 and 4000 g·mol⁻¹, preferably comprised between 300 and 1400 g·mol⁻¹, more preferentially comprised between 400 and 800 g·mol⁻¹.

Advantageously, the fatty acid comprised in the polyethylene glycol fatty acid ester has a carbon-containing chain comprising between 8 and 24 carbon atoms, preferably, between 16 and 20 carbon atoms.

A particularly preferred polyethylene glycol fatty acid ester according to the invention is polyethylene glycol-600 mono-oleate, such as that marketed by OLEON NV under the trade mark RADIA® 7404.

Throughout the present application, when a number is indicated behind the term “polyethylene glycol-” or “PEG-”, this number corresponds to the molar mass of said polyethylene glycol.

Advantageously, the composition according to the invention also comprises at least one free fatty acid and/or at least one triglyceride.

By “free fatty acid”, is meant any fatty acid molecule that is not bound to another molecule. By “fatty acid», is meant any fatty acid molecule bound to another molecule, for example when this fatty acid molecule is present in a triglyceride or in a MEL.

The at least one free fatty acid and/or at least one triglyceride can have been introduced concomitantly with the at least one MEL.

In fact, depending on the process for obtaining the MELs, such as the fermentation process described above, and in particular depending on the separation method(s) implemented in the recovery step, the latter can comprise one or more free fatty acid(s) and/or triglyceride(s).

For example, the quantity of free fatty acid(s) and/or of triglyceride(s) present in the composition according to the invention can be comprised between 0.001 and 15% by weight, preferably between 0.01 and 10% by weight, with respect to the total weight of the composition.

More particularly, the composition comprises at least one free fatty acid and at least one triglyceride. In this case, the quantity of free fatty acid(s) and triglyceride(s) present in the composition according to the invention can be comprised between 0.001 and 15% by weight, preferably between 0.01 and 10% by weight, more preferentially between 0.1 and 5% by weight, with respect to the total weight of the composition.

Advantageously, the free fatty acid(s) comprise a carbon-containing chain comprising between 14 and 24 carbon atoms, preferably 16 or 18 carbon atoms.

Advantageously, the triglyceride(s) comprise fatty acids comprising a carbon-containing chain comprising between 14 and 24 carbon atoms, preferably 16 or 18 carbon atoms.

More particularly, in the present application, and in particular in the examples, when the MELs, at the end of the recovery step, comprise at least one free fatty acid, at least one triglyceride, water and/or strains, this mixture is called “mixture of MELs”.

In this case, the free fatty acid(s) and/or triglyceride(s) can originate from the residual oil present with the MEL(s) at the end of the fermentation process described above, said residual oil being the oil utilized as a carbon source in the fermentation process, which has not been used by the strains. In addition, the free fatty acid(s) can originate from the metabolism, by the strains, of the triglycerides comprised in the oil utilized as a carbon source in said process.

Moreover, according to the process for obtaining MELs, such as the fermentation process described above, and in particular according to the separation method(s) implemented in the recovery step, the MELs can also comprise water and fungal strains, more particularly yeast strains.

According to a preferred embodiment of the composition according to the invention, it comprises a mixture of MELs having the following characteristics:
a content of MELs greater than or equal to 40% by weight, preferably greater than or equal to 50% by weight, more preferentially greater than or equal to 55% by weight;
a content of other components less than or equal to 60% by weight, preferably less than or equal to 50% by weight, more preferentially less than or equal to 45% by weight (including free fatty acids, triglycerides, water and/or strains),
   the percentages by weight being given with respect to the total weight of the mixture of MELs.

Advantageously, in this preferred embodiment, the content of water and/or strains is less than 3% by weight, with respect to the total weight of the mixture of MELs.

This mixture of MELs can in particular be obtained according to the fermentation process described above.

An example of a mixture of MELs and the process for obtaining it is also described in the following publication:
“Downstream processing of mannosylerythritol lipids produced by Pseudozyma aphidis” Rau et al.; European Journal of Lipids Science and Technology 107 (2005) 373-380.

Preferably, a mixture of MELs comprises MELs of different classes, in general at least MELs-A, B and C. Preferentially, this mixture of MELs comprises MELs-A, B, C and D.

Moreover, a mixture of MELs advantageously comprises MELs-A and MELs-B at a content comprised between 50 and 90% by weight, preferably comprised between 60 and 85% by weight, the percentages by weight being indicated with respect to the weight of the total quantity of MELs.

In addition, a mixture of MELs advantageously comprises MELs-C at a content greater than or equal to 5% by weight, preferably greater than or equal to 10% by weight, the percentages by weight being indicated with respect to the weight of the total quantity of MELs.

More particularly, a mixture of MELs can comprise MELs-A and MELs-B at a content comprised between 60% and 75% by weight, and MELs-C at a content greater than or equal to 20% by weight, the percentages by weight being indicated with respect to the weight of the total quantity of MELs.

Such mixtures of MELs are for example obtained using a fermentation process such as those described above.

It is also possible to obtain a mixture of MELs having a content of MELs greater than or equal to 95%, preferably greater than or equal to 98% by weight, with respect to the total weight of the mixture of MELs. This mixture of MELs can, for example, be obtained using the fermentation process described above to which a purification step is added, at the end of the recovery step. This purification step can comprise a liquid/liquid extraction and/or passing over a mineral substrate. Passing over a mineral substrate can be a chromatography, such as an adsorption chromatography on a silica column, carried out using suitable solvents. Such solvents are known to a person skilled in the art.

According to a preferred alternative embodiment of the composition according to the invention, it can therefore also comprise a mixture of MELs which has the following characteristics:
a content of MELs greater than or equal to 95% by weight, preferably greater than or equal to 98% by weight,
   the percentages by weight being given with respect to the total weight of the mixture of MELs.

Moreover, the purification step, following the recovery step of the MELs, can be carried out so as to obtain one class of MELs or even one MEL, at a content greater than or equal to 50%. By way of example, this purification step can comprise a liquid/liquid extraction and/or passing over a mineral substrate (such as a chromatography), as defined above.

According to a first embodiment of a composition according to the invention, it is a phytosanitary composition and comprises a pesticide active ingredient.

Advantageously, the pesticide active ingredient is selected from the following active ingredients: herbicides, fungicides, insecticides, acaricides, algicides, growth regulators, insect repellents, biocontrol and/or plant defence stimulators.

Preferably, the pesticide active ingredient is an herbicide active ingredient, a fungicide active ingredient, an insecticide active ingredient and/or a plant defence stimulator active ingredient.

Advantageously, the phytosanitary composition according to the invention comprises:
one or more fungicide active ingredients such as a carboxamide, a strobilurin, an azole (triazole, imidazole), a heterocyclic compound (pyridine, pyrimidine, piperazine, morpholine), a carbamate, an essential oil (cinnamaldehyde, thymol, tea oil), a micro-organism (fungi such as Gliocladium catenulatum, yeasts, bacteria such as Bacillus subtilis) other than those capable of being comprised in the mixture of MELs described above, a polysaccharide (chitosan) and/or,
one or more herbicide active ingredients such as a lipid biosynthesis inhibitor, an acetolactase synthase inhibitor (also called “ALS inhibitor”), a photosynthesis inhibitor, an acetamide, a derivative of amino acids such as an organophosphorus-containing derivative of amino acid (glufosinate or glyphosate) or salts thereof (ammonium salts of glufosinate, mono or di ammonium, potassium, isopropylamine salts of glyphosate), an aryloxyphenoxyproprionate, bipyridyl, cyclohexanedione, a dinitroaniline, diphenyl ether, hydroxybenzonitrile, imidazolinone, a phenoxy acetic acid, pyrazine, pyridine, a sulphonylurea, a triazine, a urea, a carbamate, a fatty acid of natural origin having an herbicide activity (caprylic acid, pelargonic acid) or derivatives thereof (salts, soaps), and/or
one or more insecticide active ingredients such as an organo(thio)phosphate, a carbamate, a pyrethroid, a growth regulator of insects, an agonist/antagonist of the nicotinic receptors, an antagonist of GABA, a macrocyclic lactone, geraniol, eugenol, thymol, neem oil, and/or
one or more plant defence stimulator active ingredients, such as Bacillus subtilis, a derivative of jasmonic acid, an alga extract.

The phytosanitary composition according to the invention can be stored for a long time before use, without reducing its pesticide active ingredient content. In fact, the active ingredient content in the composition according to the invention reduces very little over time, even after storage for several months. This effect is more fully described in Example 7.

Advantageously, the pesticide active ingredient is of natural origin. Such pesticide active ingredients are generally called bio-pesticide active ingredients or biocontrol active ingredients.

Moreover, it will be noted that the active ingredient comprised in the phytosanitary composition according to the invention can have at the same time several of the following properties: herbicide, fungicide, insecticide, acaricide, growth regulator, insect repellent and/or plant defence stimulator.

Advantageously, the quantity of pesticide active ingredient is comprised between 0.1 and 30% by weight, preferably between 1 and 20% by weight, more preferentially between 5 and 15% by weight with respect to the total weight of the phytosanitary composition.

The present application relates more particularly to a composition according to the invention, in combination with glyphosate or one of the salts thereof, and/or glufosinate or one of the salts thereof, and/or cinnamaldehyde, and/or Bacillus subtilis, and/or Gliocladium catenulatum.

Preferably, the composition according to the invention is in combination with glyphosate or one of the salts thereof and/or glufosinate or one of the salts thereof.

According to a second embodiment of a composition according to the invention, it is a cosmetic composition and comprises a cosmetic active ingredient.

Advantageously, the cosmetic composition comprises one or more cosmetic active ingredient(s) selected from:
a moisturizer such as jojoba oil, sweet almond oil, paraffin, wheat germ oil, collagen, pectin, chitosan, a glycosaminoglycan, and/or
an organic UV filter such as PABA, PARA, a salicylate, a cinnamate, an anthranilate, benzophenone-3, butyl methoxydibenzoylethane, ethylhexyl triazone, dometrizol trisiloxane, diethylhexyl butamido triazone, 4-methylbenzylidene camphor, bemotrizinol, diethylamine hydroxybenzoyl hexyl benzoate, phenyl salicylate, methylene bis-benzotriazolyl tetramethylbutylphenol, benzophenone-1, benzophenone-2, benzophenone-8, bis-ethylhexyloxyphenol methoxy-phenyl triazine, or a mineral UV filter, and/or
an anti-ageing agent such as a retinoid, an α- or β-hydroxy acid, a water-soluble vitamin, ascorbyl palmitate, a ceramide, a pseudo ceramide, a phospholipid, cholesterol, a sterol and/or,
an anti-cellulite agent such as isobutylmethyixanthine, theophylline, and/or
an anti-acne agent such as resorcinol, resorcinol acetate, benzoyl peroxide, salicylic acid, azelaic acid, and/or
a firming agent such as a plant extract (linseed extract), rose water, and/or
a vitamin such as vitamin A, derivatives thereof, vitamin B2, pantothenic acid, vitamin D, vitamin E.

Advantageously the quantity of cosmetic active ingredient is comprised between 0.1 and 30% by weight, preferably between 0.5 and 20% by weight, more preferentially between 1 and 15% with respect to the total weight of the cosmetic composition.

Moreover, a composition according to the invention can be used in various other applications, such as in fire pumps.

The invention also relates to a process for the preparation of a composition according to the invention, comprising a step of mixing at least one fatty acid methyl or ethyl ester, at least one mannosylerythritol lipid and at least one non-ionic surfactant having an HLB value greater than or equal to 12.

The compositions according to the invention are easy to prepare, by simple mixing of the components.

Advantageously, the mixing is carried out at ambient temperature under normal temperature and pressure (NTP) conditions.

Preferably, during the mixing, the components are heated at a temperature comprised between 25 and 55° C., more preferentially between 30 and 50° C., even more preferentially between 35 and 45° C.

Heating the components can allow a better homogenization of the composition according to the invention.

Advantageously, the process for the preparation of a composition according to the invention comprises a step of mixing at least one fatty acid methyl or ethyl ester, at least one mannosylerythritol lipid, at least one non-ionic surfactant having an HLB value greater than or equal to 12 and at least one alcohol having between 1 and 16 carbon atoms.

Optionally, prior to mixing, the process for the preparation of a composition according to the invention comprises obtaining at least one MEL, such as it is described above.

Advantageously, the MEL(s) are as described above and can be obtained by the fermentation process of MEL(s) described above, optionally followed by a purification step.

Advantageously, the fatty acid methyl or ethyl ester(s) and the non-ionic surfactant(s) having an HLB value greater than or equal to 12 used in the process have the characteristics of these components as they are described above.

According to a first embodiment of the process for the preparation of a composition according to the invention, it is a process for the preparation of a phytosanitary composition and comprises a step of mixing at least one fatty acid methyl or ethyl ester, at least one mannosylerythritol lipid, and at least one non-ionic surfactant having an HLB value greater than or equal to 12, with a pesticide ingredient.

According to a second embodiment of the process for the preparation of a composition according to the invention, it is a process for the preparation of a cosmetic composition and comprises a step of mixing at least one fatty acid methyl or ethyl ester, at least one mannosylerythritol lipid, and at least one non-ionic surfactant having an HLB value greater than or equal to 12, with a cosmetic active ingredient.

Advantageously, the pesticide active ingredient or the cosmetic active ingredient used in the two embodiments described above have the characteristics of these components as they are described above.

Moreover, the invention relates to an emulsion comprising a composition according to the invention and water.

The compositions according to the invention can in fact be used as an emulsifiable concentrate.

When an emulsion is prepared from the composition according to the invention, it is stable over time. By “stable”, is meant that the emulsion shows no or shows little separation of phases after storage for 30 seconds, preferably 30 minutes, more preferentially 2 hours, even more preferentially 24 hours. Advantageously, the stability of an emulsion prepared from the composition according to the invention is characterized according to step (ii) of the standard CIPAC MT 36.3.

Moreover, an emulsion prepared from the composition according to the invention does not comprise or comprises little foam.

The stability of emulsions prepared from the composition according to the invention is more fully described in Example 3.

In addition, it has been demonstrated by the inventors that, surprisingly, an emulsion comprising the composition according to the invention has a high wetting power. Thus, when an emulsion comprising the composition according to the invention is applied to a solid surface (such as a hydrophobic flat surface), the wetting of this solid surface by this emulsion is high (see Examples 4 and 10).

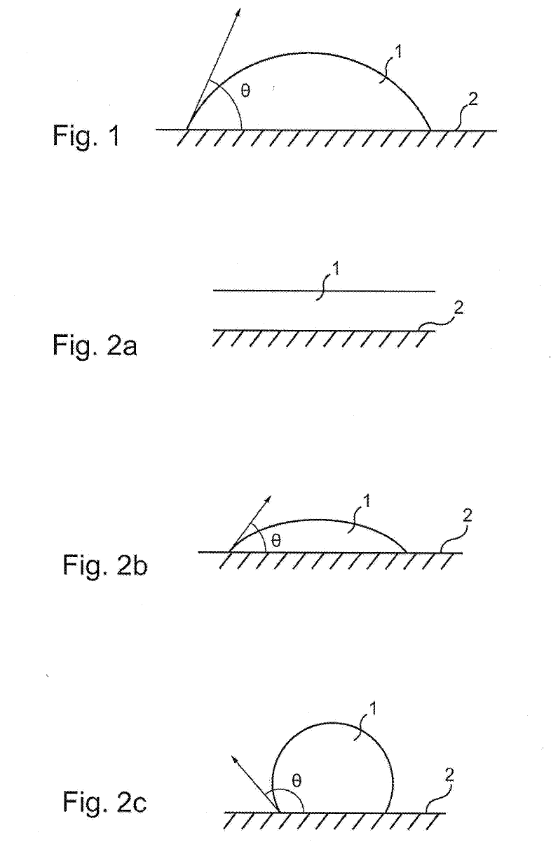

In the context of the present application:
by “wetting”, is meant the spreading ability of a liquid over a solid;
by “surface tension” of a liquid, is meant the force exerted at the interface between this liquid and a solid.
by contact angle of a drop of liquid 1 deposited on a flat solid surface 2, is meant the angle θ formed by the tangent to the drop of liquid 1 at the point of contact with the flat solid surface 2, as shown in FIG. 1.

By way of example, when a liquid, such as a drop of solution or of emulsion, and a solid, such as a plant wall or leaf, are placed in contact, the ability of the liquid to wet the solid, i.e. to spread or become distributed over it, will depend directly of the force exerted at the interface between liquid and solid, which is generally defined as the surface tension. The surface tension therefore represents the force making it possible for the liquid to adhere to the solid, or preventing it becoming distributed over it. Thus the higher surface tension, the less the liquid is capable of wetting the solid in question.

Several cases in point can therefore be shown in order to illustrate the idea of wetting. FIGS. 2a to 2c represent more particularly three cases in point.

Thus, as shown in FIG. 2a, when a drop of liquid 1 falls onto a flat solid surface 2, it can achieve a total wetting of this surface 2, i.e. become distributed over the entire surface thereof, by forming a film with a contact angle θ equal to 0 with said surface 2. Alternatively, the drop 1 can partially wet the surface 2 (FIG. 2b), i.e. it does not become totally distributed over the latter, by forming a drop with a contact angle θ comprised between 0 and 90° with said surface 2. Finally, the drop 1 may not wet the surface 2 at all (FIG. 2c), i.e. it does not become distributed over it, by forming a drop with a contact angle θ greater than 90° with said surface 2.

Moreover, an emulsion comprising the composition according to the invention has a high penetrating power. This characteristic is more particularly described in Example 10.

Thus, a phytosanitary emulsion prepared from a composition according to the invention will have an effectiveness, or an improved effectiveness, relative to a phytosanitary emulsion not comprising such a composition, as demonstrated in Example 11.

Moreover, an emulsion comprising the composition according to the invention has the advantage of being able to be sprayed in the form of droplets with a diameter advantageously greater than 100 μm, which makes it possible to reduce the phenomenon of drift during spraying.

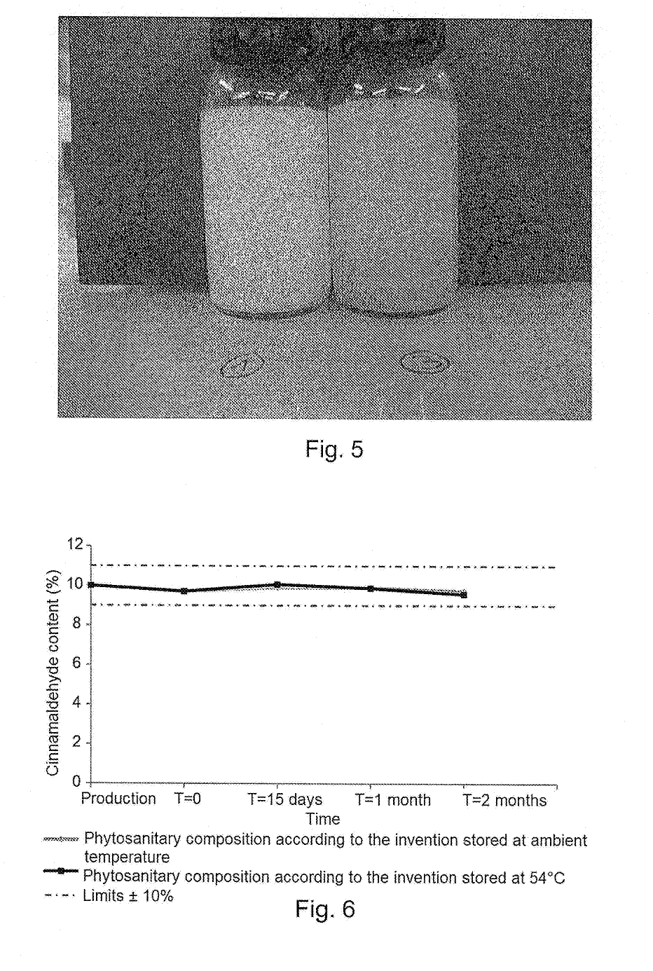

Moreover, an emulsion comprising the composition according to the invention has a white colour which is particularly favourable for its use in various fields, such as for example in cosmetics. This characteristic is more particularly demonstrated in FIG. 5.

The water is chosen depending on the use envisaged for the emulsion, or on the nature of the composition from which the emulsion is prepared.

For example, in the context of the preparation of an emulsion from a phytosanitary composition, the water is of the type of that used in the phytosanitary field, such as a drill water, which can be a water having a hardness from medium to hard. Advantageously, the water having a hardness from medium to hard has a hardness comprised between 300 and 600 ppm, preferentially between 450 and 550 ppm. Such an emulsion is generally intended to be sprayed, for example by a farmer on crops.

In the context of the preparation of an emulsion from a cosmetic composition, the water is a water generally used in cosmetics, such as distilled water or a water treated by reverse osmosis.

Advantageously the quantity of composition according to the invention in the emulsion according to the invention is comprised between 0.01 and 20% by weight, preferably between 0.05 and 10% by weight, even more preferentially between 0.1 and 5% by weight, with respect to the total weight of the emulsion.

Preferably, the quantity of water in the emulsion according to the invention is comprised between 50 and 99.99% by weight, preferably between 80 and 99.9% by weight, more preferentially between 85 and 99.9% by weight, with respect to the total weight of the emulsion.

In particular, the quantity of water can be comprised between 90 and 99.5% by weight, with respect to the total weight of the emulsion.

Advantageously, the emulsion according to the invention is an oil-in-water emulsion.

The invention also relates to a process for the preparation of an emulsion according to the invention, comprising a step of mixing a composition according to the invention with water.

Advantageously, during the mixing step, the components require gentle stirring.

The invention also relates to the use of a composition according to the invention as adjuvant.

Preferably, the adjuvant has a wetting and/or penetrating property.

The invention will be better understood in light of the examples which follow, given by way of illustration, with reference to the following figures:

FIG. 1, which represents the contact angle θ formed by the tangent to a drop of liquid 1 at the point of contact with a flat solid surface 2;

FIG. 2, which represents three cases in point showing the idea of wetting, namely the case of a total wetting of a flat solid surface 2 by a drop of liquid 1 (FIG. 2a), the case of a partial wetting of a flat solid surface 2 by a drop of liquid 1 (FIG. 2b), and the case where a drop of liquid 1 does not wet a flat solid surface 2 (FIG. 2c);

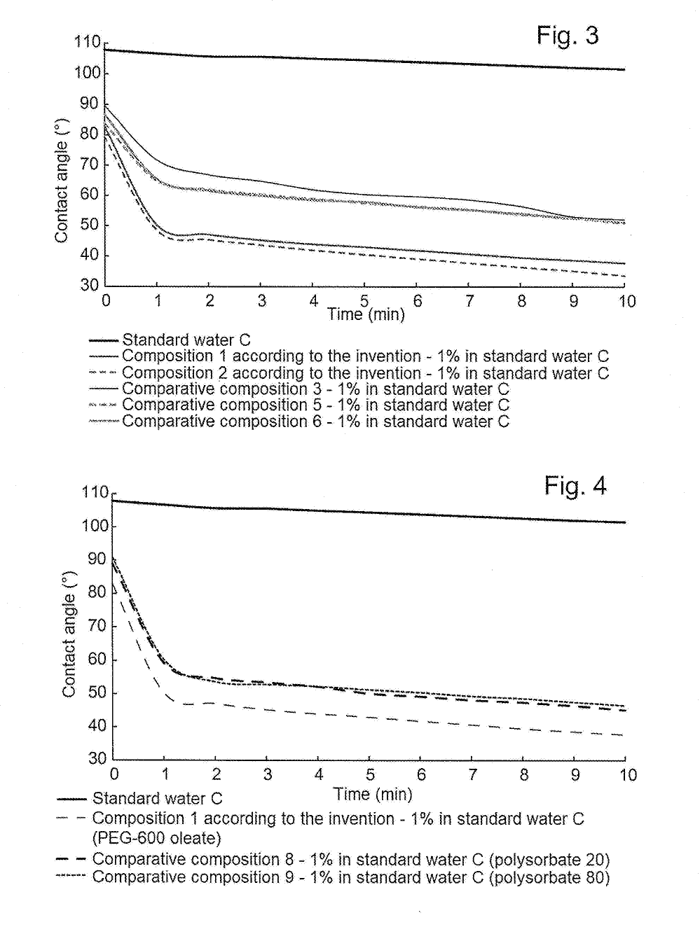

FIG. 3 which is a diagram representing the reduction in the contact angle obtained with emulsions comprising a composition according to the invention and emulsions comprising comparative compositions;

FIG. 4, which is a diagram representing the reduction in the contact angle obtained with emulsions comprising different non-ionic surfactants having an HLB value greater than or equal to 12;

FIG. 5, which is a photograph of an emulsion comprising a composition according to the invention (bearing the number 1 in the photograph) and of an emulsion comprising a comparative composition (bearing the number 3 in the photograph);

FIG. 6, which is a diagram representing the change in the content of pesticide active ingredient over time of a phytosanitary composition according to the invention;

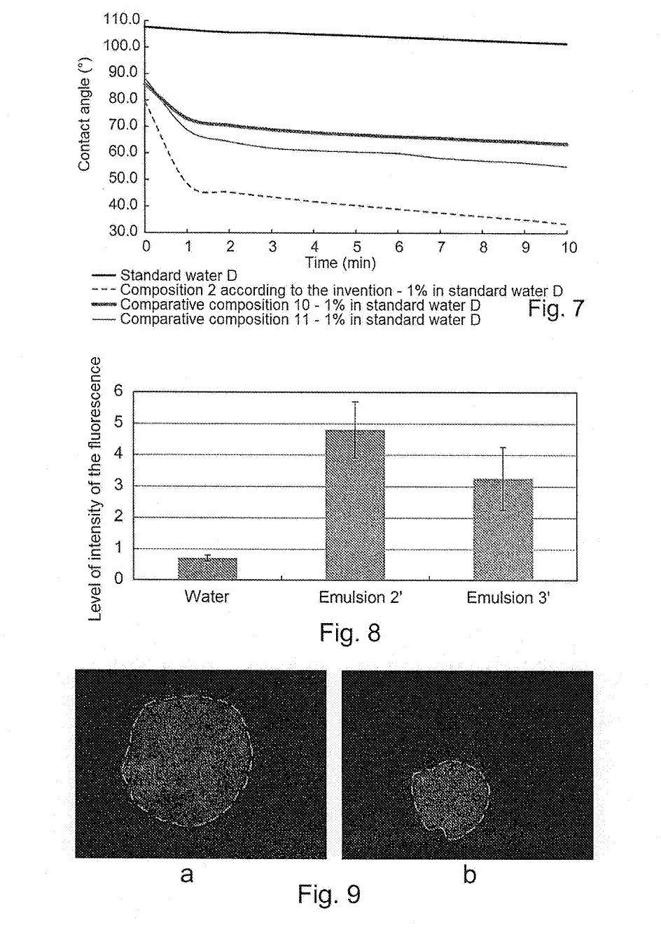

FIG. 7, which is a diagram representing the reduction in the contact angle obtained with an emulsion prepared from a composition according to the invention, and with emulsions comprising comparative compositions;

FIG. 8, which is a diagram representing the intensity of the fluorescence emitted by the cells of plants treated with a control (water), with an emulsion prepared from a composition according to the invention and with an emulsion comprising a comparative composition;

FIG. 9, which comprises two photographs representing respectively the intensity of the fluorescence emitted by the cells of plants treated with an emulsion prepared from a composition according to the invention (photograph a) and with an emulsion comprising a comparative composition (photograph b);

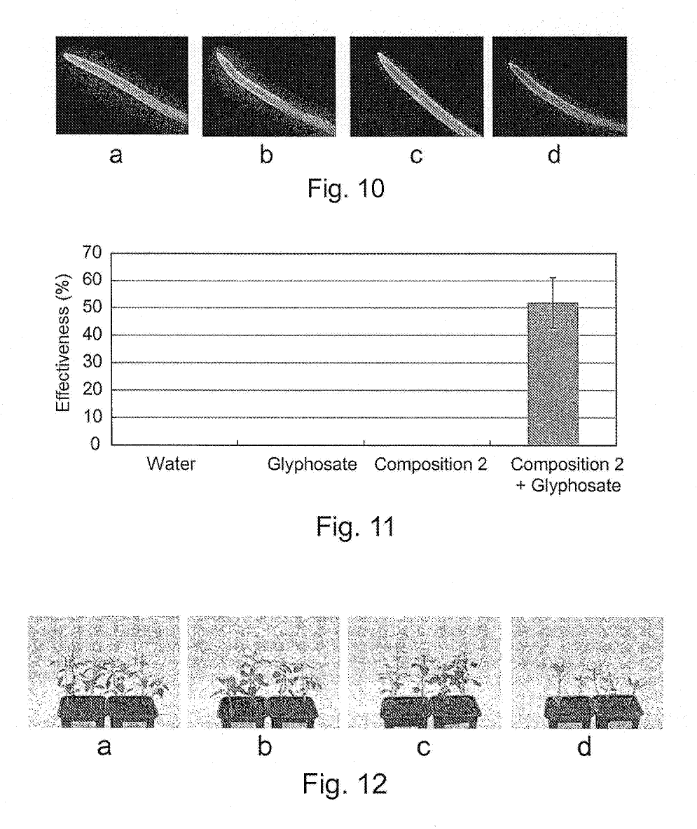

FIG. 10, which comprise four photographs representing respectively the intensity of the fluorescence (24 to 48 h after treatment) emitted by the cells of a plant root treated with an emulsion comprising a composition according to the invention and glyphosate (photograph d), with a control solution (water) (photograph a), with a solution of glyphosate alone (photograph b) and with an emulsion comprising a composition according to the invention alone (photograph c);

FIG. 11, which is a diagram representing the effectiveness of a treatment of plants with an emulsion comprising a composition according to the invention and glyphosate, with a control solution (water), with a solution of glyphosate alone and with an emulsion comprising a composition according to the invention alone;

FIG. 12, which comprises four photographs representing respectively plants treated with an emulsion comprising a composition according to the invention and glyphosate (photograph d), with a control solution (water) (photograph a), with a solution of glyphosate alone (photograph b) and with emulsion comprising a composition according to the invention alone (photograph c).

### EXAMPLE 1

### Preparation of a Composition According to the Invention

1. Obtaining the Mas

The MELs were obtained by a fermentation process comprising the following steps:
culturing a yeast strain such as Pseudozyma aphidis in the presence of vegetable oil (rapeseed) in order to obtain the MELs; and
recovering the MELs thus obtained.

At the end of the step of recovering the MELs, a mixture of MELs is obtained, which has the following characteristics:
Content of MELs: 55% by weight
Content of other components: 45% by weight (of which 42% by weight free fatty acids and triglycerides and 3% by weight water and strain),
   the percentages by weight being given with respect to the total weight of the mixture of MELs obtained.

In particular, the mixture of MELs comprise MELs-A at a content of 52% by weight, MELs-B at a content of 12% by weight, MELs-C at a content of 35% by weight, and MELs-D at a content of 1% by weight, the percentages by weight being given with respect to the weight of the total quantity of MELs. 2. Fatty Acid Methyl Esters

Radia® 7955 from OLEON NV was used.

Radia® 7955 is composed of fatty acid methyl esters of rapeseed oil (“EMC”).

3. Alcohol

2-octanol from Sigma-Aldrich was used.

4. Non-ionic Surfactant having an HLB Value Greater than or Equal to 12

Polyethylene glycol-600 mono-oleate (“PEG-600-oleate”) marketed by OLEON NV under the trade mark RADIA® 7404 was used (HLB: 13.2).

5. Process for the Preparation of Compositions According to the Invention

Composition 1

4% by weight the mixture of MELs, 93% by weight Radia® 7955 and 3% by weight Radia® 7404 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Composition 2

4% by weight the mixture of MELs, 90% by weight Radia® 7955, 3% by weight 2-octanol and 3% by weight Radia® 7404 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

### EXAMPLE 2

### Evaluation of the Emulsifiability of Compositions According to the Invention and Comparative Compositions

1. Materials and Methods

1.1 Materials

The products that were used in this example are the following:
compositions 1 to 2 according to the invention prepared in Example 1
Actirob B® (phytosanitary adjuvant based on methyl esters of rapeseed oil fatty acids, comprising a mixture of ionic and non-ionic surfactant(s), marketed by OLEON NV)
the mixture of MELs prepared in Example 1
methyl esters of rapeseed oil fatty acids (Radia® 7955, OLEON NV)
polyethylene glycol-600 mono-oleate (Radia® 7404, OLEON NV, HLB: 13.2)
2-octanol (Sigma-Aldrich)
standard water C (prepared according to the standard CIPAC MT 18.1.3)

The following equipment was also used in this Example:
60 mL glass flasks
100 mL graduated test tubes
5 mL graduated pipettes

1.2. Methods

Compositions According to the Invention

Compositions 1 and 2 prepared in Example 1 were used.

Preparation of the Comparative Compositions

Comparative Composition 3

This composition comprises only Actirob B.

Comparative Composition 4

2% by weight of the mixture of MELs and 98% by weight of Actirob B® were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Comparative Composition 5

94% by weight of Radia® 7955, 3% by weight of 2-octanol and 3% by weight of Radia® 7404 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Comparative Composition 6

97% by weight of Radia® 7955 and 3% by weight of Radia® 7404 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Comparative Composition 7

96% by weight of Radia® 7955 and 4% by weight of the mixture of MELs were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Evaluation of the Emulsifiability of Compositions 1 to 7

A protocol for the preparation of emulsions from compositions 1 and 2 according to the invention and from comparative compositions 3 to 7 was carried out, according to step (i) of the standard CIPAC MT 36.3 (“CIPAC method 2000. Prepared by the German Formulation Panel (DAPF). Chairman: G Menschel.”).

1% by weight of the different compositions 1 to 7 were respectively added to 99% by weight standard water in 100 mL graduated test tubes. The graduated test tubes were closed using a stopper. The graduated test tubes were then turned over once. According to note 4 of the standard CIPAC MT 36.3, when a test tube is “turned over once”, it means that the test tube is turned over manually so as to turn through an angle of 180° C., then returned to its initial position, the operation taking approximately two seconds.

After 30 seconds, it was observed with the naked eye whether an emulsion had formed or not in each test tube, When an emulsion had formed, it was observed whether it was uniform (complete), or on the other hand incomplete. When a uniform emulsion forms, this means that the composition has an excellent emulsifiability. On the other hand, when the emulsion is incomplete, this means that the composition does not have a good emulsifiability.

The results are presented in Table 1 below.

TABLE 1
Emulsifiability of compositions 1 to 7

| | Observation at |
|---|---|
| Composition added to standard water | 30 seconds |
| Composition 1 according to the invention | Uniform emulsion |
| (4% mixture of MELs, 93% EMC, 3% PEG-600- | |
| oleate) | |
| Composition 2 according to the invention | Uniform emulsion |
| (4% mixture of MELs, 90% EMC, 3% 2-octanol, | |
| 3% PEG-600-oleate) | |
| Comparative composition 3 | Uniform emulsion |
| (Actirob B ®) | |
| Comparative composition 4 | No emulsion formed |
| (2% mixture of MELs, 98% Actirob B ®) | |
| Comparative composition 5 | Uniform emulsion |
| (94% EMC, 3% 2-octanol, 3% PEG-600-oleate) | |
| Comparative composition 6 | Uniform emulsion |
| (97% EMC, 3% PEG-600-oleate) | |
| Comparative composition 7 | No emulsion formed |
| (96% EMC, 4% mixture of MELs) | |

The results presented in Table 1 show that the compositions according to the invention, when they are added to water, allow the formation of uniform emulsions, and this occurs spontaneously (after gentle stirring: a single manual turning). The compositions according to the invention therefore have an excellent emulsifiability.

Emulsions 1 to 3, 5 and 6 prepared from compositions 1 to 3, 5 and 6 were used in Example 3 hereafter.

### EXAMPLE 3

### Evaluation of the Stability of Emulsions Prepared from Compositions According to the Invention and Comparative Compositions

The stability of emulsions 1 to 3, 5 and 6 prepared in Example 2 was evaluated, according to step (ii) of the standard CIPAC MT 36.3. When an emulsion is stable, it appears in the form of a single phase. On the other hand, when an emulsion is unstable, a separation of the oil phase and the aqueous phase can be observed with the naked eye. This separation of phases results in the presence of cream, which represents the aqueous phase during separation, and free oil, which represents the oil phase during separation. The level of separation of the oil phase and of the aqueous phase can be quantified by the volumes of cream and of free oil present in the emulsion. Moreover, the preparation of an emulsion can result in the formation of foam. On an industrial scale, the drawback of the formation of foam during the preparation of a phytosanitary emulsion is that the user is not able to pour the quantity of water required for the preparation of this emulsion, which can result in too high a concentration of phytosanitary active ingredient, or even in an overflowing of the tank containing the emulsion. The quantity of foam can be quantified by the volume of foam present in the emulsion.

Evaluation of the Stability of Emulsions 1 to 3, 5 and 6

Following observation of emulsions 1 to 3, 5 and 6 of Example 2 for 30 seconds, the test tubes containing these emulsions were turned over ten times then deposited in a room where they remained for 24 hours at a constant temperature of 20+/−2° C. At 30 minutes, at 1 hour, at 2 hours and at 24 hours, the volumes of free oil and/or of cream formed at the top or at the bottom of the emulsions, were measured by reading the corresponding volume on the graduated test tubes. The volumes of foam were also measured in the same way.

Results

The results are presented in the Tables 2 and 3, hereafter.

TABLE 2
Stability of emulsions 1 and 2 according to the invention

| | Emulsion 1 | | | Emulsion 2 | | |
|---|---|---|---|---|---|---|
| | Foam | Cream | Oil | Foam | Cream | Oil |
| | (mL) | (mL) | (mL) | (mL) | (mL) | (mL) |
| 30 minutes | 1 | 0 | 0 | 1 | 0 | 0 |
| 1 hour | 1 | 0 | 0 | 0.5 | 0 | 0 |
| 2 hours | 0 | 1 | 0.2 | 0.5 | 1 | 0 |
| 24 hours | 0 | 0 | 1 | 0 | 1 | 0 |

TABLE 3
Stability of comparative emulsions 3, 5 and 6

| | Comparative emulsion 3 | | | Comparative emulsion 5 | | | Comparative emulsion 6 | | |
|---|---|---|---|---|---|---|---|---|---|
| | Foam | Cream | Oil | Foam | Cream | Oil | Foam | Cream | Oil |
| | (mL) | (mL) | (mL) | (mL) | (mL) | (mL) | (mL) | (mL) | (mL) |
| 30 minutes | 0 | 0.5 | 0 | 3 | 0.5 | 0 | 3 | 0.5 | 0 |
| 1 hour | 0 | 0.5 | 0 | 2 | 1 | 0 | 2 | 1 | 0 |
| 2 hours | 0 | 1 | 0 | 1 | 1 | 0 | 1 | 1 | 0 |
| 24 hours | 0 | 2 | 0 | 0 | 2 | 0 | 0 | 2 | 0 |

The results presented in Tables 2 and 3 show that emulsions 1 and 2 according to the invention have a better stability than comparative emulsions 3, 5 and 6. In particular, the appearance of cream in emulsions 1 and 2 according to the invention is less rapid than in comparative emulsions 3, 5 and 6.

Moreover, emulsions 1 and 2 according to the invention comprise less foam than comparative emulsions 3, 5 and 6.

In addition, by comparing emulsions 1 and 2, it is noted, after 24 h, that oil appears on the surface of emulsion 1 not comprising alcohol. This phenomenon is not observable for emulsion 2, which is of identical composition to emulsion 1 but which also comprises an alcohol. As a result, the latter allows better stabilization of the emulsion over time.

Moreover, an emulsion comprising a composition according to the invention has a very white colour, which makes it particularly suitable for a cosmetic use.

By way of example, the photographs of emulsion 1 comprising composition 1 according to the invention (bearing the number 1 in the photograph) and of emulsion 6 comprising comparative composition 6 (bearing the number 3 in the photograph) are shown in FIG. 5. These photographs were taken 5 minutes after turning the test tubes containing these emulsions ten times.

### EXAMPLE 4

### Evaluation of the Wetting Power of Emulsions Prepared from Composition s According to the Invention and from Comparative Compositions

1. Materials and Methods

1.1. Materials

Emulsions 1 to 2 according to the invention, and comparative emulsions 3, 5 and 6 prepared in Example 2 were used. A control solution of standard water was also prepared.

The following equipment was used:
glass flasks,
1 mL syringe provided with a needle with a diameter of 0.63 mm
a hydrophobic parafilm (Parafilm “M”, NEENAH, Wis. 54956)
the goniometer DSA10 (KRUSS)
the “Drop shape analysis” software (KRUSS)

2. Methods

Measurement of the Contact Angles

Measurements of the contact angle were carried out for each of emulsions 1.2, 3, 5, 6 and for the control solution, using the goniometer.

To this end, a drop of each emulsion and of the control solution (3 μL) was formed using the syringe. The syringe was then placed approximately 0.5 cm above the hydrophobic parafilm. By gravity, this drop became detached from the needle and fell onto the hydrophobic parafilm. The variation in the contact angle was monitored for 10 minutes from the moment when the drop touched the parafilm, using the analysis software.

The results were then processed in order to compare the variations in contact angles for each of emulsions 1 to 3, 5 and 6 with respect to the control solution.

2. Results

The results of the measurements of the contact angle for each of emulsions 1 to 3, 5 and 6 and for the control solution are presented in FIG. 3.

The results show that the reduction in the contact angle obtained with emulsions 1 and 2 according to the invention is greater than that obtained with each of comparative emulsions 3, 5 and 6, Emulsions 1 and 2, comprising the composition according to the invention, therefore have a wetting power greater than that of emulsions 3, 5 and 6, comprising the comparative compositions.

### EXAMPLE 5

### Effect of Different Non-ionic Surfactants Having an HLB Value Greater Than or Equal to 12 on the Wetting Power of Emulsions Comprising Them

3. Materials and Methods

3.1. Materials

The following products were used:
the mixture of MELs prepared in Example 1
methyl esters of rapeseed oil fatty acids (Radia® 7955, OLEON NV)
polyethylene glycol-600 mono-oleate (Radia® 7404, OLEON NV)
polysorbate 20 (Radia® 7137, OLEON NV, HLB: 16.5)
polysorbate 80 (Radia® 7157, OLEON NV, HLB: 14.9)
standard water C

The following equipment was used:
glass flasks,
a 1 mL syringe provided with a needle with a diameter of 0.63 mm
a hydrophobic parafilm (Parafilm “M”, NEENAH, Wis. 54956)
the goniometer DSA10 (KRUSS)
the “Drop shape analysis” software (KRUSS)

4. Methods

Preparations of the Compositions

Composition 1 according to the invention was used.

Composition 8 according to the invention

4% by weight of the mixture of MELs, 93% by weight of Radia® 7955, and 3% by weight of polysorbate 20 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Composition 9 according to the invention

4% by weight of the mixture of MELs, 93% by weight of Radia® 7955, and 3% by weight of polysorbate 80 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Preparations of the Emulsions

Emulsions 1, 8 and 9 comprising respectively compositions 1 (emulsion 1), 8 (emulsion 8) and 9 (emulsion 9), were prepared according to step (i) of the standard CIPAC MT 36.3, as indicated hereafter.

In flasks, 1% by weight of each of the compositions 1, 8 and 9 was respectively added to 99% by weight of standard water. The flasks were closed using a stopper. The flasks were then turned once in order to obtain emulsions 1, 8 and 9.

A control solution of standard water was also prepared.

Measurement of the Contact Angles

Measurements of the contact angle were carried out for each of emulsions 1, 8, 9 and for the control solution, using the goniometer.

To this end, a drop of each emulsion or of the control solution (3 μL) was formed using the syringe. The syringe was then placed approximately 0.5 cm above the hydrophobic parafilm. By gravity, this drop became detached from the needle and fell onto the hydrophobic parafilm. The variation in the contact angle was monitored for 10 minutes from the moment when the drop touched the parafilm, using the analysis software.

The results were then processed in order to compare the variations in contact angles for each of emulsions 1, 8 and 9, with respect to the control solution.

2. Results

The results of the measurements of the contact angle for each of emulsions 1, 8, 9 and for the control solution are presented in FIG. 4.

The results show that the reduction in the contact angle obtained with emulsion 1 is greater than that obtained with each of emulsions 8 and 9.

### EXAMPLE 6

### Use of a Phytosanitary Composition According toe the Invention in the Preparation of an Emulsion

A phytosanitary composition according to the invention was prepared then used in the preparation of an emulsion.

The phytosanitary composition according to the invention has the following characteristics:

| Components | % by weight* |
|---|---|
| Mixture of MELs | 4% |
| Radia ® 7955 | 79.95% |
| Radia ® 7404 | 3% |
| 2-octanol | 3% |
| Cinnamaldehyde (Herbarom laboratoire) | 10% |
| Eugenol (Sigma-Aldrich) | 0.05% |

*Percentage by weight with respect to the total weight of the phytosanitary composition.

The emulsion was prepared as follows:

50% by weight of water, 1% by weight of the phytosanitary composition according to the invention, then 49% by weight of water were successively added into a resealable container, the percentages by weight being indicated with respect to the total weight of the phytosanitary composition-water mixture obtained. The container was then sealed then turned over in order to obtain the emulsion. If necessary, the pH and the salinity of the water will have been adjusted beforehand.

The emulsion according to the invention obtained has the following characteristics:

| Components | % by weight* |
|---|---|
| Phytosanitary composition according to the | 1% |
| invention | |
| Water | 99% |

*Percentage by weight with respect to the total weight of the emulsion.

### EXAMPLE 7

### Evaluation of the Content of Pesticide Active Ingredient over Time in a Phytosanitary Composition According to the Invention

The change in the cinnamaldehyde content over time of the phytosanitary composition according to the invention prepared in Example 6 was analysed by gas chromatography (GC analysis).

1. Equipment

The following equipment was used:
a balance accurate to 0.00001 g
1 and 5 mL automatic pipettes
Pasteur pipettes
20 and 100 mL measuring flasks
a chromatograph for gas chromatography (GC 6580—Agilent Technologies)
a 5 μL syringe

2. Methods

The phytosanitary composition according to the invention prepared in Example 6 was stored for two months during which a GC analysis was carried out at different times (production, 0 days, 15 days, 1 month, 2 months, 3 months).

The storage was carried out:
at ambient temperature (approximately 20° C. under normal temperature and pressure (NTP) conditions), or
at 54° C.

The parameters of the GC analysis are the following:
injector: split mode; temperature: 350° C.; split ratio: 10:1
mobile phase: Helium
carrier gas flow rate: 2 mL/min
analysis time: 36 minutes
injection volume: 2 μL
detector: FID; temperature: 250° C.; air: 450 mL/min; H2: 40 mL/min; He: 30 mL/min
column: DB5 HT 15 m*250 μm*0.1 μm

All the components used in the preparation of the phytosanitary composition according to the invention were passed alone through GC according to the GC parameters above. This made it possible to verify that none of these components had a retention time identical to cinnamaldehyde or to the internal standard (azulene).

More particularly:
the retention time of cinnamaldehyde is 6 min,
the retention time of azulene is 6.7 min.
the components have retention times which are different to those of cinnamaldehyde and azulene.

The selectivity of the method is in compliance.

During a 1^{st} step, the internal calibration with solutions of cinnamaldehyde at different concentrations was carried out.

Then, the 2^{nd} step consisting of the GC analysis was carried out, followed by calculation of the level of cinnamaldehyde in the phytosanitary composition, using the calibration curve.

The results of the GC analysis are presented in Table 4 below and in FIG. 6.

TABLE 4
Results of the GC analysis of the phytosanitary
composition according to the invention

| | | Cinnamaldehyde content | | | |
|---|---|---|---|---|---|
| | | Ambient | | | Limits max |
| Time | | temperature | 54° C. | Limits min 10% | 10% |
| Production | | 10 | 10 | 9 | 11 |
| 0 | days | 9.71 | 9.71 | 9 | 11 |
| 15 | days | 9.91 | 10.07 | 9 | 11 |
| 1 | month | 9.89 | 9.88 | 9 | 11 |
| 2 | months | 9.74 | 9.57 | 9 | 11 |
| 3 | months | 10.22 | 9.81 | 9 | 11 |

The results show that the cinnamaldehyde content of the phytosanitary composition according to the invention reduces very slightly over time, whatever the storage conditions.

### EXAMPLE 8

### Use of a Cosmetic Composition According to the Invention in the Preparation of an Emulsion

A cosmetic composition according to the invention was prepared then used in the preparation of an emulsion.

The cosmetic composition according to the invention has the following characteristics:

| Components | % by weight* |
|---|---|
| Mixture of MEL(s) | 2% |
| Methyl olivate | 93% |
| (PEL-IS ® OME - Elé corporation) | |
| Radia ® 7404 | 1.5% |
| Lauric alcohol | 1.5% |
| Jojoba oil | 2% |

*Percentage by weight with respect to the total weight of the cosmetic composition.

The emulsion was prepared as follows:

50% by weight of water, 5% by weight of the composition according to the invention, and 45% by weight of water were successively added into a flask, the percentages by weight being indicated with respect to the total weight of the phytosanitary composition-water mixture obtained. The flask was then turned over in order to obtain the emulsion. If necessary, the pH and the salinity of the water will have been adjusted beforehand.

The emulsion according to the invention obtained has the following characteristics:

| Components | % by weight* |
|---|---|
| Cosmetic composition according to the | 5% |
| invention | |
| Water | 95% |

*Percentage by weight with respect to the total weight of the emulsion.

Other components can be added to this emulsion, such as other cosmetic active ingredients and/or formulation agents, the latter making it possible in particular to confer desired texture (cream, gel) and/or sensory properties on it.

### EXAMPLE 9

### Evaluation of the Stability and of the Wetting Power of Emulsions Prepared from a Composition According to the Invention and from Comparative Compositions

1. Materials and Methods

1.1. Materials

The following products were used:
Composition 2 according to the invention prepared in Example 1
polyglycerol-3 caprylate/caprate (PG-3-C8/C10, C094, OLEON)
Simulsol® SL11W (SEPPIC)
methyl esters of rapeseed oil fatty acids (“EMC”, Radia® 7955, OLEON NV)
2-octanol (Sigma-Aldrich)
polyethylene glycol-600 mono-oleate (Radia® 7404, OLEON)
standard water D (prepared according to the standard CIPAC MT 18.1.4)

The following equipment was used:
glass flasks,
graduated test tubes,
graduated pipettes
a 1 mL syringe provided with a needle with a diameter of 0.63 mm
a hydrophobic parafilm (Parafilm “M”, NEENAH, Wis. 54956)
the goniometer DSA10 (KRUSS)
the “Drop shape analysis” software (KRUSS)

1.2. Methods

Preparation of the Composition According to the Invention

Composition 2 of Example 1 was used.

Preparation of Comparative Compositions 10 and 11

Comparative Composition 10:

4% by weight of polyglycerol-3 caprylate/caprate, 90% by weight of Radia® 7955, 3% by weight of 2-octanol and 3% by weight of Radia® 7404 were added into a 120 mL glass flask, the percentages by weight being indicated with respect to the total weight of the composition obtained, then stirred manually until homogenization of the composition was achieved. During stirring, the composition is heated at approximately 50° C. in order to facilitate the homogenization.

Comparative Composition 11:

4% by weight of Simulsol® SL11W, 90% by weight of Radia® 7955, 3% by weight of 2-octanol and 3% by weight of Radia® 7404 were added into a 120 mL glass flask, the percentages by weight being indicated with respect to the total weight of the composition obtained, then stirred manually until homogenization of the composition was achieved. During stirring, the composition is heated at approximately 50° C. in order to facilitate the homogenization.

It is observed that comparative compositions 10 and 11 are cloudy at ambient temperature, as well as after heating at 50° C. In addition, for these two compositions, a deposit is observed at the bottom of the flask 24 hours after stirring. On the other hand, composition 2 according to the invention is homogeneous at ambient temperature, as well as after heating at 50° C. No deposit is observed at the bottom of the flask 24 hours after stirring.

Preparation of Emulsions from Compositions 2, 10 and 11

A protocol for the preparation of emulsions from composition 2 according to the invention and from comparative compositions 10 and 11 was carried out, according to step (i) of the standard CIPAC MT 36.3 (“CIPAC method 2000. Prepared by the German Formulation Panel (DAPF). Chairman: G Menschel.”).

1% by weight of the different compositions 2, 10 and 11 were respectively added to 99% by weight of standard water in graduated test tubes, the percentages by weight being indicated with respect to the total weight of the water-composition mixture. The flasks were closed using a stopper. The flasks were then turned once.

After 30 seconds, it could be observed with the naked eye that a uniform emulsion had formed from the comparative composition 2. Non-uniform (incomplete) emulsions had formed from the comparative compositions 10 and 11.

Evaluation of the Stability of Emulsions 2, 10 and 11

The stability of prepared emulsions 2, 10 and 11 was evaluated, according to step (ii) of the standard CIPAC MT 36.3.

Following observation of emulsions 2, 10 and 11 at 30 seconds, the test tubes containing these emulsions were turned over ten times then deposited in a room where they remained for 24 hours at a constant temperature of 20+/−2° C. At 30 minutes, at 1 hour, at 2 hours and at 24 hours, the volumes of free oil and/or of cream formed at the top or at the bottom of the emulsions, were measured by reading the corresponding volume on the graduated test tubes. The volumes of foam were also measured in the same way.

Results

The results are presented in Table 5 hereafter.

TABLE 5
Stability of emulsions 2, 10 and 11

| | Emulsion 2 | | | Emulsion 10 | | | Emulsion 11 | | |
|---|---|---|---|---|---|---|---|---|---|
| | Foam | Cream | Oil | Foam | Cream | Oil | Foam | Cream | Oil |
| | (mL) | (mL) | (mL) | (mL) | (mL) | (mL) | (mL) | (mL) | (mL) |
| 30 minutes | 1 | 0 | 0 | 3 | 1 | 0 | 2 | 0.5 | 0.5 |
| 1 hour | 1 | 0 | 0 | 1 | 1 | 0 | 1 | 1 | 1 |
| 2 hours | 0 | 1 | 0.2 | 1 | 1 | 0 | 1 | 1 | 1 |
| 24 hours | 0 | 0 | 1 | 0 | 2 | 0 | 0 | 2 | 2 |

The results presented in Table 5 show that emulsion 2 according to the invention has a better stability than comparative emulsions 10 and 11. In particular, the appearance of cream in emulsion 2 according to the invention is less rapid than in comparative emulsions 10 and 11, and it can be observed that the cream is no longer present in emulsion 2 at 24 hours.

2. Evaluation of the Wetting Power of Emulsions of 2, 10 and 11

Measurement of the Contact Angles

Measurements of the contact angle were carried out for each of emulsions 2, 10 and 11 and for a control solution of standard water D, using the goniometer.

To this end, a drop of each emulsion or of the control solution (3 μL) was formed using the syringe. The syringe was then placed approximately 0.5 cm above the hydrophobic parafilm. By gravity, this drop became detached from the needle and fell onto the hydrophobic parafilm. The variation in the contact angle was monitored for 10 minutes from the moment when the drop touched the parafilm, using the analysis software.

The results were then processed in order to compare the variations in contact angles for each of emulsions 2, 10 and 11, with respect to the control solution.

Results

The results of the measurements of the contact angle for each of emulsions 2, 10 and 11 and for the control solution are presented in FIG. 7.

The results show in particular that the reduction in the contact angle obtained with emulsion 2 is noticeably greater than that obtained with each of emulsions 10 and 11.

### EXAMPLE 10

### Evaluation of the Penetrating Power and of the Wetting Power of Emulsions Prepared from a Composition According to the Invention and from a Comparative Composition

This example is based on the use of a fluorochrome emitting a green fluorescence during its entry into plant cells. Thus, a direct visualization of the penetrating power and of the wetting power of a product to be tested can be carried out using this fluorochrome. The quantification of the intensity of the fluorescence is carried out by image analysis using suitable software. This makes it possible to quantify the level of penetration of the fluorochrome.

1. Materials and Methods

1.1. Materials

The following products were used:
composition 2 according to the invention prepared in Example 1
comparative composition 3 prepared in Example 2
water
a fluorochrome
chenopodium plants

1.2. Methods.

Compositions 2 and 3 were prepared 15 minutes before treatment of the chenopodium plants, in order to ensure a good homogeneity thereof.

Composition 3 comprises only Actirob B®, which is an adjuvant known for its wetting and penetrating properties.

A control solution comprising only water was also prepared.

Preparation of Emulsions 2′ and 3′ from Compositions 2 and 3

Emulsions 2′ and 3′ were prepared by mixing 0.25% by weight of composition 2 or 3 with 99.75% by weight of standard water, the percentages by weight being indicated with respect to the total weight of the water-composition mixture.

The fluorochrome was added to each of emulsions 2′ and 3′ and to the control solution.

The fluorochrome was added 2 minutes before treatment of the chenopodium plants.

Evaluation of the Fluorescence

Emulsions 2′ and 3′ and the control solution were respectively deposited on chenopodium plants, and the fluorescence was evaluated.

The intensity of the fluorescence is representative of the penetrating power of the emulsions.

The size of the surface on which the fluorescence is visible is representative of the wetting power of the emulsions.

The results are presented in FIGS. 8 and 9.

In these FIGS. 8 and 9, it is noted that the intensity of the fluorescence emitted by the cells of the plants treated with emulsion 2′ is high, which is evidence of the penetrating power of emulsion 2′ comprising a composition according to the invention.

Moreover, it can be observed in FIG. 9 that the fluorescence is emitted over a larger surface by the cells of plants treated with the emulsion comprising composition 2 according to the invention (FIG. 9a) than by the cells of plants treated with the emulsion comprising comparative composition 3 (FIG. 9b). The wetting power of the emulsion prepared from the composition 2 according to the invention is therefore greater than that of the emulsion prepared from comparative composition 3.

### EXAMPLE 11

### Composition According to the Invention in Combination with a Pesticide Active Ingredient

Composition 2 according to the invention prepared in Example 1 was used in combination with an herbicide active ingredient (glyphosate) in the treatment of chenopodium plants (Chenopodium album). The chenopodium plants used were at a 2 to 3 leaves stage (BBCH 12-13), and were grown in a phytotron at a temperature of 15° C. during the night and 20° C. during the day, with a photoperiod of 14 hours. The glyphosate used was non-formulated (technical ingredient, reference 4521, Sigma-Aldrich®). The glyphosate is known to be not very effective or ineffective when it is used alone as it does not adhere to the leaves and penetrates with difficulty.

Four treatments were carried out:
a control treatment (control), during which 500 g/ha of water supplemented with 2% by weight of fertilizing agent is sprayed on the plants,
a treatment with glyphosate alone, during which 500 g/ha of a solution at 0.25% by weight of glyphosate supplemented with 2% by weight of fertilizing agent is sprayed on the plants,
a treatment with composition 2 according to the invention, during which 500 g/ha of an emulsion at 0.25% by weight of composition 2 according to the invention and supplemented with 2% by weight of fertilizing agent is sprayed on the plants,
a treatment with glyphosate and composition 2 according to the invention, during which an emulsion at 0.25% by weight of glyphosate and 0.25% by weight of composition 2 according to the invention and supplemented with 2% by weight of fertilizing agent is sprayed on the plants.

The treatments were carried out using a Teejet® XR1100015 nozzle at 200 l/ha.

Each treatment is carried out on 4 different pots, each pot containing 4 plants.

The different treatments carried out are presented in Table 6 below.

TABLE 6
Treatments carried out in Example 11.

| | | Ammonium |
|---|---|---|
| | | sulphate |
| | | (fertilizing |
| Treatment | dose | agent) |
| Control solution (water) | 500 g/ha | 2% |
| Solution at 0.25% of glyphosate | 500 g/ha | 2% |
| Emulsion at 0.25% of composition 2 | 500 g/ha | 2% |
| according to the invention | | |
| Emulsion at 0.25% of glyphosate and 0.25% | 500 g/ha | 2% |
| composition 2 according to the invention | | |

The percentages are the percentages by weight with respect to the total weight of solution or of emulsion.

The evaluation of the effectiveness was carried out by measuring the cell activity of the roots of the plants 24 to 48 h after treatment. The cell activity is measured by staining the cells using fluorescent markers making it possible to distinguish:
the cells having a metabolic activity (living cells), which emit a green fluorescence;
the structures which emit a blue fluorescence; and
the dead cells, which emit a red fluorescence.

When the cell metabolic activity reduces, the green fluorescence reduces. The results are presented in FIG. 10.

A reduction can be noted in the intensity of the fluorescence in the cells of the roots of plants treated with the emulsion at 0.25% by weight of glyphosate and 0.25% by weight of composition 2 according to the invention (FIG. 10d). This reduction is not observed for the other treatments with the control solution (FIG. 10a), the solution at 0.25% by weight of glyphosate alone (FIG. 10b) and the emulsion at 0.25% by weight of composition 2 according to the invention (FIG. 10c).

Evaluation of the effectiveness was also carried out by observation of the plants with the naked eye 14 days after treatment. The effectiveness is evaluated in percentage of destruction of the vegetation, taking into account the size of the plants and the symptoms of phytotoxicity such as deformation of the leaves, the appearance of white patches and necrosis.

The results are presented in Table 7 below and in FIGS. 11 and 12.

TABLE 7
Average effectiveness of the treatments carried out in Example 11.

| | | Ammonium | |
|---|---|---|---|
| | | sulphate | |
| | | (fertilizing | Effectiveness |
| Treatment | dose | agent) | (average*) |
| Control solution (water) | 500 g/ha | 2% | 0 |
| Solution of glyphosate | 500 g/ha | 2% | 0 |
| Emulsion at 0.25% of | 500 g/ha | 2% | 0 |
| composition 2 according to the | | | |
| invention | | | |
| Emulsion at 0.25% of | 500 g/ha | 2% | 51.9 +/− 9.2 |
| glyphosate and 0.25% | | | |
| composition 2 according to the | | | |
| invention | | | |

The percentages are the percentages by weight with respect to the total weight of solution or emulsion.
*The results represent the average effectiveness for each treatment, calculated on the basis of the results obtained for the 4 different pots comprising 4 chenopodium plants.

The results of Table 7 and of FIGS. 11 and 12 show that the phytotoxicity of the emulsion at 0.25% by weight of glyphosate and 0.25% by weight of composition 2 according to the invention is high (FIG. 11 and FIG. 12d). This phytotoxicity is not observed for the other treatments with the control solution (FIG. 11 and FIG. 12a), the solution at 0.25% by weight of glyphosate alone (FIG. 11 and FIG. 12b) and the emulsion at 0.25% by weight of composition 2 according to the invention (FIG. 11 and FIG. 12c).

Thus, a composition according to the invention can be used in combination with a pesticide active ingredient.

### EXAMPLE 12

### Evaluation of the Emulsifiability of Comparative Compositions Comprising a Non-ionic Surfactant Having an HLB Value of less than 12

1. Materials and Methods

1.1 Equipment

The products that were used in this example are the following:
the mixture of MELs prepared in Example 1
methyl esters of rapeseed oil fatty acids (Radia® 7955 OLEON NV)
polyethylene glycol-600 di-oleate (Radia® 7444, OLEON NV, HLB: 10)
polyethylene glycol-200 mono-oleate (Radia® 7402, HLB: 7)
2-octanol (Sigma-Aldrich)
standard water D (prepared according to the standard CIPAC MT 18.1.4)

The following equipment was also used in this example:
60 mL glass flasks
100 mL graduated test tubes
5 mL graduated pipettes

1.2. Methods

Preparation of Comparative Compositions

Comparative Composition 12

4% by weight of the mixture of MELs, 90% by weight of Radia® 7955, 3% by weight of 2-octanol and 3% by weight of Radia® 7444 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Comparative Composition 13

4% by weight of the mixture of MELs, 90% by weight of Radia® 7955, 3% by weight of 2-octanol and 3% by weight of Radia® 7402 were added into a 60 mL glass flask, the percentages by weight being indicated with respect to the total weight of the composition obtained, then stirred manually until homogenization of the composition was achieved. During stirring, it is possible to heat the composition at 40° C. in order to facilitate the homogenization.

Evaluation of the Emulsifiability of the Comparative Compositions 12 and 13

A protocol for the preparation of emulsions from comparative compositions 12 and 13 was carried out, according to step (i) of the standard CIPAC MT 36.3 (“CIPAC method 2000. Prepared by the German Formulation Panel (DAPF). Chairman: G Menschel”).

1% by weight of the different compositions 12 and 13 were respectively added to 99% by weight of standard water in 100 mL graduated test tubes. The graduated test tubes were closed using a stopper. The graduated test tubes were then turned once.

1. A composition comprising:
between 75 and 99% by weight of at least one fatty acid methyl or ethyl ester,
between 0.01 and 20% by weight of at least one mannosylerythritol lipid (MEL), and
between 0.01 and 20% by weight of at least one non-ionic surfactant having an HLB value greater than or equal to 12,
the percentages by weight being indicated with respect to the total weight of the composition.

2. The composition according to claim 1, comprising between 0.1 and 20% by weight of at least one alcohol having a number of carbon atoms comprised between 1 and 16, with respect to the total weight of the composition.

3. The composition according to claim 1, in which the total quantity of non-ionic surfactant(s) having an HLB value greater than or equal to 12 is comprised between 0.5 and 10% by weight, with respect to the total weight of the composition.

4. The composition according to claim 1, in which the total quantity of MEL(s) is comprised between 0.1 and 10% by weight, with respect to the total weight of the composition.

5. The composition according to claim 1, comprising at least two MELs selected from the group consisting of MEL-A, MEL-B, MEL-C and MEL-D.

6. The composition according to claim 1, in which the fatty acid methyl or ethyl ester is a methyl or ethyl ester of rapeseed, soya and/or olive oil fatty acid.

7. The composition according to claim 1, in which the non-ionic surfactant having an HLB value greater than or equal to 12 is a polyethylene glycol fatty acid ester.

8. The composition according to claim 1, further comprising at least one free fatty acid and/or at least one triglyceride.

9. The composition according to claim 1, further comprising a pesticide active ingredient.

10. The composition according to claim 1, further comprising glyphosate or one of the salts thereof, and/or glufosinate or one of the salts thereof, and/or cinnamaldehyde, and/or Bacillus subtilis, and/or Gliocladium catenulatum.

11. The composition according to claim 1, further comprising a cosmetic active ingredient.

12. A process for the preparation of a composition according to claim 1, comprising mixing at least one fatty acid methyl or ethyl ester, at least one mannosylerythritol lipid and at least one non-ionic surfactant having an HLB value greater than or equal to 12.

13. An emulsion comprising a composition according to claim 1 and water.

14. A process for the preparation of an emulsion, comprising mixing a composition according to claim 1 with water.

15. A method for protecting a plant comprising treating a plant with the composition of claim 9.

16. A method for improving the wetting and/or penetrating property of a pesticide or cosmetic active ingredient comprising mixing the pesticide or cosmetic active ingredient with the composition of claim 1.
